(19) United States
(12) Plant Patent Application Publication
(10) Pub. No.: US 2016/0205832 P1
(43) Pub. Date: Jul. 14, 2016

(54) White delight two

(50) ;

(71) Applicant: The Texas A&M University System, College Station, TX (US)

(72) Inventors: David H. Byrne, Bryan, TX (US); Natalie Anderson, Calvert, TX (US)

(73) Assignee: The Texas A&M University System, College Station, TX (US)

(21) Appl. No.: 14/544,507

(22) Filed: Jan. 13, 2015

(51) Int. Cl. A01H 5/00

(52) U.S. Cl. PLT/195

(57) ABSTRACT

Disclosed is a new variety of Prunus persica named ‘WHITE DELIGHT TWO’. This new variety, which requires approximately 500-550 chilling units of dormancy, is considered to be a peach tree of early mid-season maturity, which is productive and produces white-fleshed, sub-acid fruit that are very firm, attractively colored, and suitable for both local and long-distance shipping.

### BACKGROUND OF THE INVENTION

### Field of the Invention

This invention relates to peach trees and, more specifically, to peach trees referred to as a variety of Prunus persica named ‘WHITE DELIGHT TWO’. ‘WHITE DELIGHT TWO’, which requires approximately 550 chilling units of dormancy, produces an exceptionally high quality, firm, sub-acid, semi-freestone, white-fleshed peach that matures in mid-June in the medium chill zone of Texas.

### SUMMARY OF THE INVENTION

The ‘WHITE DELIGHT TWO’ peach is characterized as to novelty and is otherwise noteworthy by producing fruit that ripens in the early mid-season; is considered high quality, subacid, white-fleshed peach which is firm and has an attractive coloration. In this regard, the present variety of peach tree bears fruit that are ripe for commercial harvesting and shipment in mid-June, when the fruit is grown in the medium chill zone of Texas. ‘WHITE DELIGHT TWO’ ripens about two weeks after ‘June Gold’ (Brooks, 1958) and about with ‘Scarlet Pearl, an acid flavored white-fleshed peach (Okie, 1993). This new variety exhibits the potential to be commercialized in medium chill regions where ‘TexKing’ (Byrne and Bacon, 2004), ‘TexRoyal’ (Byrne and Bacon, 1991), and ‘June Gold’ are commercially grown.

### ORIGIN OF THE VARIETY

The present peach tree was the result of an ongoing Stone Fruit Breeding Program of Texas A & M University, College Station, Brazos County, Tex. To this end, both controlled and hybrid crosses are made each year in order to produce seedling populations from which improved progenies are evaluated and selected.

The seedling ‘WHITE DELIGHT TWO’ was originated at the Texas A & M University Horticultural Farm in College Station, Tex. ‘White Delight Two’ (WD2, TX3E213LW) is a cross between the white-fleshed seedling selection TX4D46W and ‘Summer Sweet’, a patented peach released by Zaiger Genetics (Zaiger, 1992). TX4D46W was a white-fleshed peach selection from a seedling population derived from a cross between ‘TexRoyal’ and a subacid white peach seedling. The parents of ‘TexRoyal’ (Byrne and Bacon, 1991) are NJ239 and ‘Early Amber’. NJ 239 was derived from an open pollinated seedling of the selection B1-75 whose parents were Tireglobe' crossed by a seedling with the parents of ‘Rio Oso Gem’ and ‘Sunbeam’. Resulting seed from this cross were planted in 2000 at the Texas A & M University Horticultural Farm in College Station, Tex. ‘WHITE DELIGHT TWO’ was marked as TX3E213LW for subsequent observation and noted as having exceptional characteristics in 2002. Two-year and older trees of the variety were subsequently evaluated during the 2005 through 2011 fruit growing seasons in both California (Fowler) and Texas (Floresville, College Station, Fairfield, and Terrell).

### ASEXUAL REPRODUCTION OF THE VARIETY

TX3E213LW was bud grafted onto virus-free Nemaguard (“The Brooks and Olmo Register of Fruit and Nut Varieties,” 3rd Ed., American Society of Horticultural Science Press, Alexandria, Va., 1997) peach rootstock in June 2002 at the nursery site in Oakdale, Calif. The variety was subsequently planted at the experimental orchard in the central portion of the San Joaquin Valley, near Fowler, Fresno County, Calif. and in three sites in Texas (College Station, Fairfield and Floresville). Fruit from the resulting propagation has been evaluated during the period from 2005 to 2011 fruit seasons. This evaluation clearly demonstrated that the re-propagated trees were true to the characteristics of the original seedling in all observable aspects.

### BRIEF DESCRIPTION OF THE DRAWINGS

This new variety of peach tree is illustrated by the accompanying photographic drawings and depicts the plant by the best possible color representation using color photography, wherein:

FIG. 1 A color photograph of a characteristic twig bearing typical leaves and several mature fruit showing their external coloration sufficiently matured for harvesting and shipment of ‘White Delight Two’.

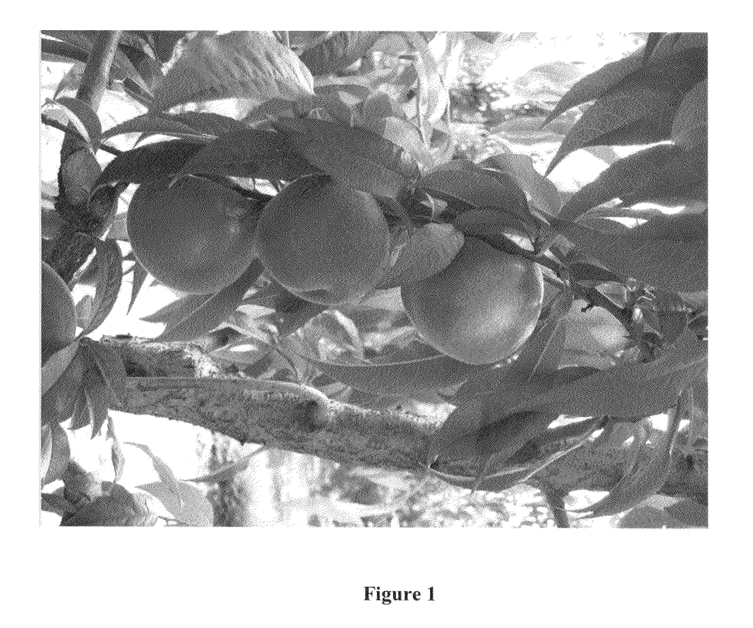

FIG. 2. ‘White Delight Two’ (right) as compared to ‘Scarlet Pearl’ (left) as grown in Fairfield, Tex. The width of the stripes are 2.54 cm (1 inch). ‘White Delight Two’ requires less chilling and is more attractive then ‘Scarlet Pearl’.

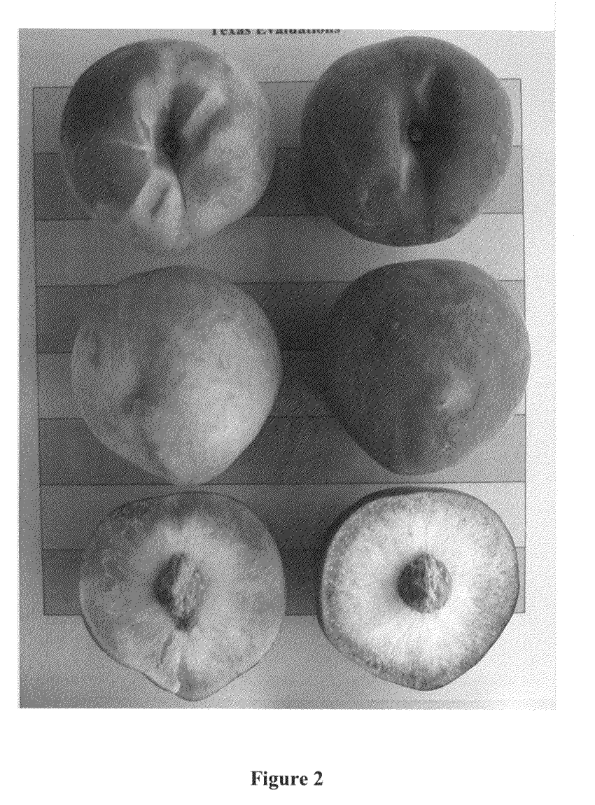

FIG. 3. ‘White Delight Two’ as grown in Fowler, Calif. showing the rounder shape of the fruit as compared to the fruit as grown in Texas (see FIG. 2).

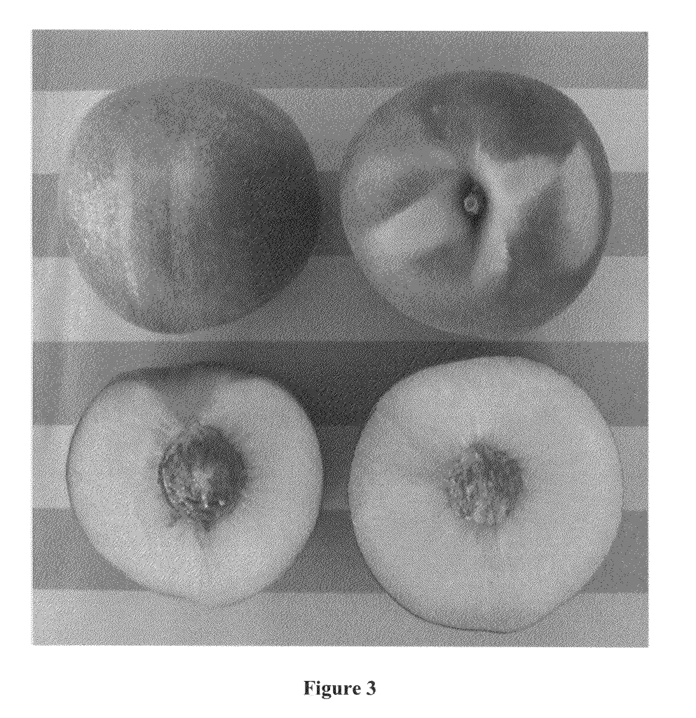

FIG. 4. Color photograph of the endocarps of ‘White Delight Two’. The ruler is demarcated in millimeters.

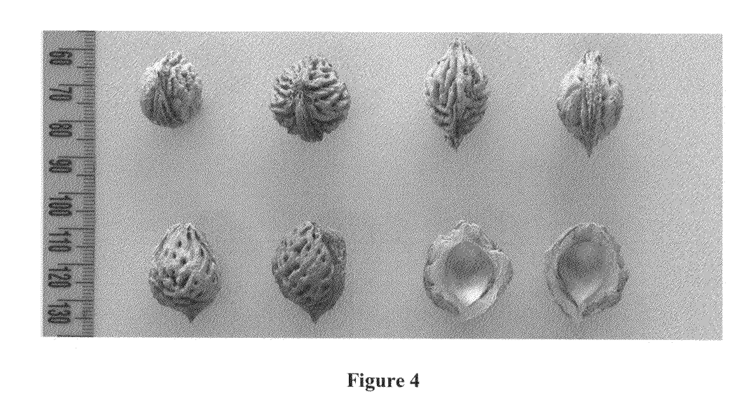

FIG. 5. The non-showy flowers of ‘White Delight Two’. The length of the upper flower is 15 mm.

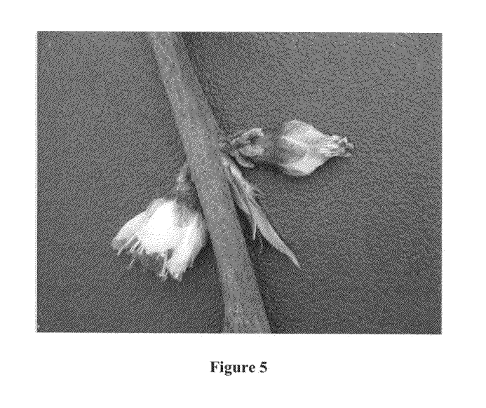

### BOTANICAL DESCRIPTION OF THE VARIETY

Referring more specifically to the pomological details of this new and distinct variety of peach tree, the following has been observed under the ecological conditions prevailing at the experimental orchards in Fowler, Calif. All major color code designations are by reference to the RHS Colour Chart (Third Edition) provided by The Royal Horticultural Society of Great Britain. Colors are approximate as color depends on horticultural practices such as light level and fertilization rate, among others.
Tree:
   Size.—Generally average to above average as compared to other common peach cultivars ripening in the early mid-season of maturity.
   Height.—8.5 feet (2.58 m) at the end of the 2010 growing season.
   Width.—6.2 feet width (1.80m) at the end of the 2010 growing season.
   Vigor.—High.
   Density.—Medium to high.
   Productivity.—Productive.
   Shape.—The ‘WHITE DELIGHT TWO’ peach has been trained in central leader configuration and exhibits a generally spreading growth habit typical of commercial varieties of peach such as “TexKing” and “TexRoyal”.
   Current season growth.—The current season growth for the new variety was approximately 3.0 to 3.3 feet (0.91-1.12 m).
   Regularity of bearing.—Regular, and considered hardy under typical central San Joaquin Valley, Calif. and central Texas conditions.
Trunk:
   Size.—Approximately 4.75 inches (12.07 cm) in diameter and 19 inches (48.26 cm) in circumference when measured at a distance of approximately 12 inches (30.5 cm) above the soil level on a five-year old tree.
   Bark texture.—Considered moderately rough with numerous folds of papery scarf-like skin being present.
   Bark coloration.—Variable, colors present are 156C-D of the Greyed-White Group, 197D of the Greyed-Green Group and N200C-D of the Brown Group.
Branches:
   Size.—Considered medium for the variety.
   Thickness.—Average (approximately 6.8 cm in diameter as measured 10 cm from the trunk on a five-year old tree) as compared to other varieties.
   Surface Texture.—Average and appearing furrowed on wood that is several years old.
   Lenticels.—Numerous flat, oval lenticels present. The lenticels range in size from approximately 3 to 10 mm in width and were approximately 1 mm in height.
   Current season shoots.—Surface texture — Substantially glabrous.
   Internode length.—Approximately 1.5 to 5.0 cm as measured in the middle of a current season stem.
   Color of mature branches.—Considered brown with grey. The predominant colors being 166A and 177A of the Greyed-Orange Group, N186C, 187A and N187D of the Greyed-Purple Group and N200D of the Brown Group.
   Current season shoots.—Color — Light to medium green (143C of the Green Group, 144A-C, 145A-B and 146B-D of the Yellow-Green Group) with some reddish-brown coloration appearing on exposed surface of the shoots (185A and N186C of the Greyed-Purple Group and 199A and N199A-C of the Grey-Brown Group).
Leaves:
   Size.—Considered moderately large for the species. Leaf measurements have been taken from vigorous upright current season growth approximately at mid-shoot.
   Leaf length.—Approximately 145 to 175 mm.
   Leaf width.—Approximately 29 to 32 mm.
   Leaf thickness.—Less than 1 mm.
   Leaf form.—Lanceolate.
   Leaf tip form.—Acuminate.
   Leaf upper surface color.—Green varying among 137A-C of the Green Group.
   Leaf lower surface color.—Green varying among 137C-D of the Green Group and 146A of the Yellow-Green Group.
   Leaf mid-vein color.—Light yellow green (149D, 150D and 154D of the Yellow-Green Group and 157C-D of the Green-White Group).
   Leaf margins.—
   Form.—Considered crenate/crenulate.
   Uniformity.—Considered generally uniform.
   Leaf petioles.—
   Size.—Considered medium long.
   Length.—Approximately 7 to 9 mm.
   Thickness.—Approximately 1 to 2 mm.
   Color.—Pale green (Yellow-Green Group 144A-C, 149D, 150D and 154D and 157C-D of the Green-White Group).
   Leaf glands.—
   Size.—Approximately 1 mm in height and width.
   Number.—Generally 0-4 per leaf.
   Type.—Reniform.
   Color.—Brown plus the underlying green of the petiole colors (199A Grey-Brown Group, 149D, 150D and 154D of the Yellow-Green Group and 157C-D of the Green-White Group).
   Leaf stipules.—
   Size.—Medium long for the species.
   Length.—Approximately 13 to 15 mm.
   Form.—Lanceolate.
   Color.—Green (Yellow-Green Group 144B-C) with reddish brown tips (Greyed-Orange Groups 174B-C and 177B) when young. The stipules are considered to be early deciduous.
   Ratio of wood (leaf) buds to flowering buds.—1 to 2 flower buds per vegetative bud.
Flowers:
   Floral buds.—
   General.—The floral buds are considered to be medium in size, conic in form, and slightly appressed relative to the bearing shoot.
   Color.—The bud scales are gray-brown, (approximately Greyed-Orange Groups 165A and 166A, Greyed-Purple Group 187A, Greyed-Green Group 198A-B, Greyed-Orange Group 177A and Grey-Brown Group N199B). The buds are considered hardy under typical central San Joaquin Valley, California and central Texas climatic conditions.
   Length.—Approximately 4 to 6 mm.
   Blooming type.—Considered medium early in relation to other peach cultivars commonly growing in the central Texas. Date of full bloom was between March 1st and March 10th with a mean full bloom date of March 6th during the period between 2006 and 2011.
   Flower type.—Small non showy.
   Flower size.—Flower diameter at full bloom is approximately 16 to 20 mm.
   Bloom quantity.—Considered abundant.
   Flower bud frequency.—Normally 1 to 2 per node.
   Petal size.—
   General.—Considered small for the species.
   Width.—Approximately 6 to 10 mm.
   Length.—Approximately 10 to 15 mm.
   Petal form.—Broadly ovate.
   Petal count.—Nearly always 5.
   Petal color.—Light to medium pink when young (White Group N155B-C, Red-Purple Group 63B-C) and darkening with advanced senescence to a deep pink (Red-Purple Groups 63C and 64D).
   Petal claw.—
   Form.—The claw is considered truncate in shape and has a medium size when compared to other varieties.
   Length.—Approximately 1 to 2 mm.
   Width.—Approximately 1 to 1.5 mm.
   Petal margins.—Generally considered variable, from nearly smooth to slightly undulate.
   Petal apex.—Generally — The petal apices appear slightly domed.
   Flower pedicel.—
   Length.—Considered short to medium-short, and having an average length of approximately 2 to 3 mm.
   Thickness.—Considered average, approximately 0.5 to 1 mm.
   Color.—A light to medium green (Yellow-Green Groups N144A-D and 146D).
   Floral nectaries.—
   Color.—Ranging in color from yellow to yellow-green (Yellow Group 3B-C and Yellow-Green 144A-B) and dull orange to orange-gold (Greyed-Orange Group 170B).
   Calyx.—
   Surface texture.—Generally glabrous.
   Color.—Highly variable from dull red to orange red with green and maroon with green (approximately Red-Purple Group 60A, Yellow-Green Group 144A-143C, Orange-Red Group 34A, Red Group 46A, Red Group 53B and Green Group 141D-143C).
   Sepals.—
   Surface texture.—The surface has a short, fine, wooly and a gray-colored texture.
   Size.—Average, and ovate in form.
   Color.—Highly variable from dull red to orange red with green and maroon with green (approximately Red-Purple Group 59A-60A, Greyed-Purple Group 187A-187B, Yellow-Green Group 144A-143C, Orange-Red Group 34A, Red Group 46A, Red Group 53B and Green Group 141D-143C).
   Anthers.—
   General.—Average in size for the species.
   Color.—Variable from yellow, orange-yellow, orange-brown to golden yellow (approximately Yellow Group 7A-7D, Yellow Group 13A, Yellow-Orange Group 17A-B, Greyed-Orange Group 165-166A and Brown Group 200A-200D).
   Pollen production.—Pollen is abundant, and is a yellow to yellow-orange color (approximately Yellow Groups 5A-B and 12A-13B and Yellow-Orange Groups 16A and 14A-15C).
   Filaments.—
   Size.—Variable in length, approximately 11 to 14 mm, with the filaments slightly shorter than the pistil.
   Color.—White with pink tinge (approximately White Group 155A-D) and darkening to pink-purple with advanced maturity (approximately Red-Purple Group 71A-B.
   Pistil.—
   General.—Average in size, but slightly longer, relative to the general anther height, overall.
   Length.—Approximately 15 to 19 mm including the ovary.
   Color.—Considered a very pale green when young (approximately Yellow Green Group 145A-147D and Green Group 138C-143C), and developing a pink-purple color with advancing senescence (approximately 184A-184B).
   Surface texture.—The variety has a long, silver white pubescent pistil (approximately White Group 155A-D).
Fruit:
   Maturity when described.—The present variety of fruit is described, as it would be found in its firm ripe condition at full commercial maturity. Under the ecological conditions prevailing in the San Joaquin Valley of Central California:
   First picking.—June 13th.
   Last picking.—June 20th.
   Size.—General Medium large to large for the season and considered uniform.
   Average cheek diameter.—Approximately 78 to 83 mm.
   Average suture diameter.—Approximately 76 to 81 mm.
   Average axial diameter.—Approximately 74 to 79 mm.
   Fruit form.—Generally round and slightly flattened axially. Also occasionally the fruit exhibits less symmetry due to a slightly protruding suture and tip. This tendency is enhanced in lower chill zones. The fruit is generally uniform in symmetry when viewed from the apical aspect.
   Fruit suture.—Generally, the suture appears as a thin line that extends from the base to the apex, and appears deeper at the apex, forming a shallow basin at the apical point. No apparent callusing or stitching exists along the suture line.
   Color.—Red Group 47A.
   Ventral surface.—Form — Generally uniform.
   Stem cavity.—Size — Considered moderately deep for the species.
   Width.—Approximately 12 to 18 mm.
   Length.—Approximately 22 to 30 mm.
   Depth.—Approximately 15 to 18 mm.
   Form.—Considered oval.
   Fruit base.—Round.
   Fruit apex.—rounded to slightly pointed.
   Fruit stem.—Length — Approximately 10 mm Thickness — 2 to 3 mm Color — Light to medium Green (Green Groups 142C-141D).
   Fruit skin.—Generally considered medium or average in thickness Surface Texture — Low to medium pubescence Skin Acidity — Considered neutral. Tenacious to Flesh — Yes at commercial maturity. Tendency to Crack — Not observed. Skin Color — Generally — Variable, with approximately 40-90% of the fruit surface covered with an attractive orange red blush. Down — Short and thick Blush Color — The blush color is generally considered dark red to medium red (Orange-Red Group N34A, Red Groups 39A-B, 46A, 47A-D and 53A and Red-Purple Group 60A) with many degrees of shading and blending between these colorations. Skin Ground Color — Very light yellow to ivory (Yellow Groups 4D, 10D and 11D and White Group 155B).
   Flesh color.—Light yellow to ivory (Yellow Group 11D and White Group 155B-C).
   Flesh fibers.—Present, numerous and lightly colored. These fibers are present throughout the flesh.
   Stone cavity color.—Light yellow to ivory (Yellow Group 11D and White Group 155A-D) with a very slight reddening coloration (Red-Purple Group 58A and Greyed-Purple Group 186A).
   Flesh texture.—Generally, the melting flesh is considered firm and fine at commercial maturity.
   Ripening.—Generally the fruit of the present variety ripens evenly.
   Flavor.—Considered very sweet and rich, with a subacid flavor.
   Aroma.—Pleasant and reasonably abundant.
   Eating.—Generally considered very good to excellent.
Stone:
   Attachment.—Semi-freestone at commercial maturity.
   Stone size.—Generally considered medium small to medium relative to the ratio of stone to fruit size.
   Length.—Approximately 27 to 34 mm.
   Width.—Approximately 17 to 18 mm.
   Thickness.—Approximately 15 to 16 mm.
   Fibers.—Generally a few medium length fibers are attached sporadically along the surface of the stone.
   Stone form.—Elliptical.
   Stone base.—Narrow.
   Apex shape.—Narrow to medium.
   Stone shape.—Considered ovoid.
   Stone surface.—
   Surface texture.—Minor surface markings are honeycombed with numerous single pits, rosettes of pits and pit grooves.
   Ridges.—Numerous ridges are present basally, and converge towards the base of the stone.
   Ventral edge.—Small to medium.
   Dorsal edge.—Shape — Grooved and having moderately rough edges.
   Stone color.—The color of the dry stone is a medium to dark brown (Greyed-Orange Groups 165A 166A-B and 177A-B and Brown Group 200D). The color of the inside surface of the endocarp is primarily Greyed-Orange 164B-D and 165B-D.
   Tendency to split.—Very slight.
   Kernel.—the kernel fills the endocarp at harvest and measures approximately 5-6 mm in thickness, 9-10 mm in width, and 12-15 mm in length. When dried the shriveled kernels measure approximately 3-4 mm in thickness, 8-10 mm in width, and 12-15 mm in length. The colors of the shriveled kernels are primarily Greyed-Orange Group 165B-D.
   Use.—The subject variety, ‘WHITE DELIGHT TWO’, is considered to be a peath tree of medium chilling requirement and early-mid season maturity, which produces subacid, white-fleshed fruit which are very firm, attractively colored, and which are useful for both local and regional market.
   Keeping quality.—Good.
   Resistance to insects and disease.—No particular susceptibilities were noted.
   Shipping quality.—Good. Although the new variety of peach tree possesses the described characteristics when grown under the ecological conditions prevailing near Fairfield, Freestone County in Texas, it will be understood that variations of the usual magnitude and characteristics incident to the changes in growing conditions, fertilization, pruning, and pest control are to be expected.

### REFERENCES

Anon. 1995. RHS Colour Chart. The Royal Hort. Soc., London.

Brooks, R. M. 1958. Double Delight, Earligold, and June Gold peaches. Fruit Var. J. 3:22.

Byrne, D. H. and T. A. Bacon. 1991. ‘TexRoyal’, a medium chilling peach. HortScience 26(10):1338-1340.

Byrne, D. H. and T. A. Bacon. 2004. ‘TexKing’, an early ripening medium chill peach. HortScience 39: 442-443.

Okie, W. R. 1993. ‘Goldprince’ and ‘Scarletpearl’ peaches. HortScience 28:231.

Zaiger, C. F., G. N. Zaiger, L. M. Gardner, and G. G. Zaiger. 1992. Peach tree ‘Summer Sweet’. U.S. Plant Pat. No. 8,070. 22 Dec. 1992.

We claim:

1. A new and distinct Prunus persica tree, substantially as illustrated and described herein.
